(19) United States
(12) Patent Application Publication
(10) Pub. No.: US 2024/0238573 A1
(43) Pub. Date: Jul. 18, 2024

(54) Shape Memory Alloy Low Profile Port

(71) Applicant: Bard Peripheral Vascular, Inc., Franklin Lakes, NJ (US)

(72) Inventors: Christian Andersen, Queen Creek, AZ (US); Bryon Ray Densley, Rochester, MN (US); Jessica Hoye, Phoenix, AZ (US); Ian N. Thomas, West Bountiful, UT (US); Diana Fiumefreddo, Salt Lake City, UT (US)

(21) Appl. No.: 18/561,683

(22) PCT Filed: May 20, 2021
(86) PCT No.: PCT/US21/33383

§ 371 Date: Nov. 16, 2023

(51) Int. Cl. A61M 39/02; A61M 39/04

(52) U.S. Cl. CPC A61M 39/0208; A61M 39/04; A61M 2205/0266

(57) ABSTRACT

Embodiments disclosed herein are directed to a port system including a body defining a reservoir and a port stem. One of the body or the stem can be formed of a shape memory material and can be transitionable between an expanded configuration and a collapsed configuration. One of the port body or the reservoir in the collapsed configuration can provide a reduced overall size or outer profile of the port for subcutaneous placement and/or between access events. The port can require a smaller incision site, required fewer stitches or no stitches at all, improving patient recovery times, patient comfort, reducing scarring and improving aesthetics. The port stem in collapsed configuration can be smaller than a catheter lumen diameter and can transition to an expanded configuration that is larger than a catheter lumen diameter facilitating a fluid tight seal therebetween.

### SUMMARY

Briefly summarized, embodiments disclosed herein are directed to a port system including a collapsible reservoir and associated methods thereof. The port can include a body defining a reservoir and formed of a shape memory material, e.g. a metal, alloy, or nitinol. The port body can transition between an expanded configuration and a collapsed configuration. Advantageously, the port can transition to the collapsed configuration to provide a reduced overall size or outer profile for insertion, and/or between access events. The port can require a smaller incision site, requiring fewer stitches or no stitches at all to close the incision site, improving patient recovery times, patient comfort, reducing scarring and improving aesthetics.

Disclosed herein is a subcutaneous access system including, a catheter defining a lumen, and a port having, a port stem configured to engage a catheter and provide fluid communication therewith, a body including a shape memory material and defining a reservoir in fluid communication with the port stem, the body transitionable between an expanded configuration and a collapsed configuration, the collapsed configuration defining a smaller overall profile, and a needle penetrable septum disposed over the reservoir and configured to provide percutaneous access thereto.

In some embodiments, the port stem includes the shape memory material and is configured to transition between an expanded configuration and a collapsed configuration. In some embodiments, the port stem in the collapsed configuration defines a first outer stem diameter, and the port stem in the expanded configuration defines a second outer stem diameter, the second outer stem diameter being larger than the first outer stem diameter. In some embodiments, the first outer stem diameter is less than an inner lumen diameter of the catheter in a relaxed state and the second outer stem diameter is greater than the inner lumen diameter of the catheter in the relaxed state. In some embodiments, the shape memory material includes a metal, composite, or an alloy and includes one of nickel, titanium, zinc, copper, gold, iron, aluminum, a copper-aluminum-nickel alloy, a nickel-titanium alloy, or nitinol.

In some embodiments, the body in the expanded configuration defines one of a first port height, a first port width, or a first port length, and wherein the body in the collapsed configuration defines one of a second port height, a second port width, or a second port length. In some embodiments, one of the second port height is less than the first port height, the second port width is less than the first port width, or the second port length is less than the first port length. In some embodiments, the body in the expanded configuration defines a first port volume, and the body in the collapsed configuration defines a second port volume, the second port volume being less than the first port volume.

In some embodiments, the reservoir in the expanded configuration defines one of a first reservoir height, a first reservoir width, or a first reservoir length, and wherein the reservoir in the collapsed configuration defines one of a second reservoir height, a second reservoir width, or a second reservoir length. In some embodiments, one of the second reservoir height is less than the first reservoir height, the second reservoir width is less than the first reservoir width, or the second reservoir length is less than the first reservoir length. In some embodiments, the reservoir in the expanded configuration defines a first reservoir volume, and the reservoir in the collapsed configuration defines a second reservoir volume, the second reservoir volume being less than the first reservoir volume.

In some embodiments, the body at a first temperature can transition from the collapsed configuration to the expanded configuration, and the body at a second temperature can transition from the expanded configuration to the collapsed configuration. In some embodiments, the first temperature is equal to or greater than 98.6° F. (37° C.) and the second temperature is less than 98.6° F. (37° C.). In some embodiments, the first temperature is greater than 98.6° F. (37° C.) and the second temperature is equal to or less than 98.6° F. (37° C.). In some embodiments, the body at the second temperature is configured to be plastically deformable. In some embodiments, the body at the first temperature is in an austenite phase, and the body at the second temperature is in a martensite phase.

In some embodiments, the body in the collapsed configuration includes a folded portion disposed on an outer surface of the body. In some embodiments, the folded portion includes a plurality of pleats disposed on an outer surface of the body and configured to allow the body to transition between the expanded configuration and the collapsed configuration. In some embodiments, the body includes one of a frame, a shell, a honeycomb or an exoskeleton morphology formed of the shape memory material. In some embodiments, the subcutaneous access system further includes a second material different from the shape memory material and disposed thereon to form a continuous outer profile.

Also disclosed is a method of manufacturing a port including, forming a body defining a reservoir and a reservoir opening, the body including a shape memory material and transitionable between an expanded configuration and a collapsed configuration, the collapsed configuration defining a smaller overall profile, forming a stem defining a stem lumen in fluid communication with the reservoir, and coupling a needle-penetrable septum with the reservoir opening, the needle penetrable septum configured to provide percutaneous access to the reservoir.

In some embodiments, the port stem includes the shape memory material and is configured to transition between an expanded configuration and a collapsed configuration. In some embodiments, the port stem in the collapsed configuration defines a first outer stem diameter, and the port stem in then expanded configuration defines a second outer stem diameter, the second outer stem diameter being larger than the first outer stem diameter. In some embodiments, the first outer stem diameter is less than an inner lumen diameter of a catheter in a relaxed state and the second outer stem diameter is greater than the inner lumen diameter of the catheter in the relaxed state. In some embodiments, the shape memory material includes a metal, composite, or an alloy and includes one of nickel, titanium, zinc, copper, gold, iron, aluminum, a copper-aluminum-nickel alloy, a nickel-titanium alloy, or nitinol.

In some embodiments, the body in the expanded configuration defines one of a first port height, a first port width, or a first port length, and wherein the body in the collapsed configuration defines one of a second port height, a second port width, or a second port length. In some embodiments, one of the second port height is less than the first port height, the second port width is less than the first port width, or the second port length is less than the first port length. In some embodiments, the body in the expanded configuration defines a first port volume, and the body in the collapsed configuration defines a second port volume, the second port volume being less than the first port volume.

In some embodiments, the reservoir in the expanded configuration defines one of a first reservoir height, a first reservoir width, or a first reservoir length, and wherein the reservoir in the collapsed configuration defines one of a second reservoir height, a second reservoir width, or a second reservoir length. In some embodiments, one of the second reservoir height is less than the first reservoir height, the second reservoir width is less than the first reservoir width, or the second reservoir length is less than the first reservoir length. In some embodiments, the reservoir in the expanded configuration defines a first reservoir volume, and the reservoir in the collapsed configuration defines a second reservoir volume, the second reservoir volume being less than the first reservoir volume.

In some embodiments, the body at a first temperature can transition from the collapsed configuration to the expanded configuration, and the body at a second temperature can transition from the expanded configuration to the collapsed configuration. In some embodiments, the first temperature is equal to or greater than 98.6° F. (37° C.) and the second temperature is less than 98.6° F. (37° C.). In some embodiments, the first temperature is greater than 98.6° F. (37° C.) and the second temperature is equal to or less than 98.6° F. (37° C.). In some embodiments, the body at the second temperature is configured to be plastically deformable. In some embodiments, the body at the first temperature is in an austenite phase, and the body at the second temperature is in a martensite phase.

In some embodiments, the body in the collapsed configuration includes a folded portion disposed on an outer surface of the body. In some embodiments, the folded portion includes a plurality of pleats disposed on an outer surface of the body and configured to allow the body to transition between the expanded configuration and the collapsed configuration. In some embodiments, the body includes one of a frame, a shell, a honeycomb, or an exoskeleton morphology formed of the shape memory material. In some embodiments, the method further includes a second material different from the shape memory material and disposed thereon to form a continuous outer profile.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a port coupled to a catheter, in accordance with embodiments disclosed herein.

FIG. 2A shows a longitudinal cross-section view of a port in an expanded configuration, in accordance with embodiments disclosed herein.

FIG. 2B shows a plan, cross-section view of a port in an expanded configuration, in accordance with embodiments disclosed herein.

FIG. 2C shows a longitudinal cross-section view of a port in a collapsed configuration, in accordance with embodiments disclosed herein.

FIG. 2D shows a plan, cross-section view of a port in a collapsed configuration, in accordance with embodiments disclosed herein.

FIG. 2E shows a plan, cross-section view of a port in an expanded configuration, in accordance with embodiments disclosed herein.

FIG. 2F shows a plan, cross-section view of a port including a folded portion and in a collapsed configuration, in accordance with embodiments disclosed herein.

FIG. 3A shows a lateral, cross-section view of a port in an expanded configuration, in accordance with embodiments disclosed herein.

FIG. 3B shows a lateral, cross-section view of a port in a collapsed configuration, in accordance with embodiments disclosed herein.

FIG. 3C shows a longitudinal, cross-section view of a port in an expanded configuration, in accordance with embodiments disclosed herein.

FIG. 3D shows a longitudinal, cross-section view of a port in a collapsed configuration, in accordance with embodiments disclosed herein.

FIG. 4A shows a longitudinal, cross-section view of a port stem in a collapsed configuration, in accordance with embodiments disclosed herein.

FIG. 4B shows a longitudinal, cross-section view of a port stem in an expanded configuration, in accordance with embodiments disclosed herein.

FIG. 4C shows a lateral, cross-section view of a port stem in a collapsed configuration, in accordance with embodiments disclosed herein.

FIG. 4D shows a lateral, cross-section view of a port stem in a collapsed configuration and including a folded portion, in accordance with embodiments disclosed herein.

FIG. 4E shows a lateral, cross-section view of a port stem in an expanded configuration, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

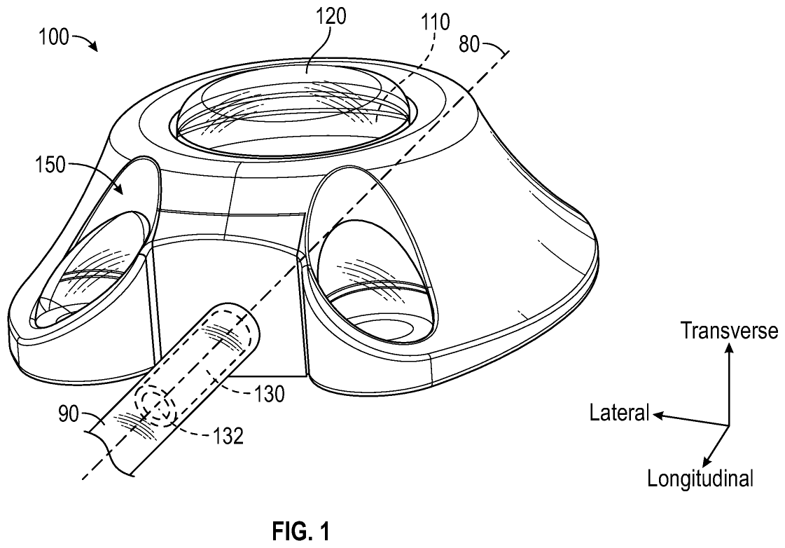

To assist in the description of embodiments described herein, as shown in FIG. 1, a longitudinal axis extends substantially parallel to an axial length of the catheter. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

FIG. 1 shows a vascular access device, or (“port”) 100 including a body including and shape memory material and transitionable between an expanded configuration and a collapsed configuration to facilitate subcutaneous placement. The port 100 can generally include a port body 150 defining a reservoir 110 and include a needle-penetrable septum 120 disposed thereover. In an embodiment, the septum 120 can be formed of a silicone rubber or similar suitable material. In use, the reservoir 110 can be accessed percutaneously by a needle penetrating the septum 120 and fluidly accessing the reservoir.

The port 100 can further include a port stem 130 extending along a stem axis 80 and defining a stem lumen 132 that is in fluid communication with the reservoir 110. In an embodiment, the stem axis 80 can extend substantially parallel to the longitudinal axis. The stem 130 can be configured to be coupled to a catheter 90 or similar device, configured to access a vasculature of a patient. The catheter 90 can include an elongate body defining a lumen 92 extending therethrough.

In an embodiment, one of the body 150 or the stem 130 can include a first material 152. In an embodiment, the first material 152 can be a shape memory material, such as a metal, alloy, alloys containing zinc, copper, gold or iron, composites, copper-aluminum-nickel alloys, nickel-titanium (NiTi) alloys (“Nitinol”), or the like. In an embodiment, one of the body 150 or the stem 130 can include a second material 154, such as a non-shape memory material, plastic, polymer, metal, alloy, composite, or the like.

In an embodiment, the body 150 can be formed entirely of the first material 152. In an embodiment, the body 150 can include a frame, shell, “honeycomb” or “exoskeleton” morphology formed of the first material 152 and can include a covering or “filler” material, formed of one or more second materials 154 disposed thereover to form a continuous outer profile. These and other configurations of materials are contemplated to fall within the scope of the present invention.

In an embodiment, the first material 152 can transition between a first configuration and a second configuration. In an embodiment, a first temperature (e.g. at or above body heat) can transition the first material 152 from the second configuration (e.g. collapsed configuration) to the first configuration (e.g. expanded configuration) and a second temperature (e.g. below body heat) can transition the first material 152 from the first configuration (e.g. expanded configuration) to the second configuration (e.g. collapsed configuration).

In an embodiment, the first material 152 at the second temperature (e.g. below body heat) can be plastically deformed from the first configuration (e.g. expanded configuration) to the second configuration (e.g. collapsed configuration). The first material 152 at the first temperature (e.g. at or above body heat) can transition the first material 152 from the second configuration (e.g. collapsed configuration) to the first configuration (e.g. expanded configuration).

In an embodiment, the first material 152 in the first (expanded) configuration can be in an austenite phase, and the first material 152 in the second (collapsed) configuration can be in a martensite phase. In an embodiment, the first material 152 at the first temperature can be in an austenite phase, and the first material 152 at the second temperature can be in a martensite phase.

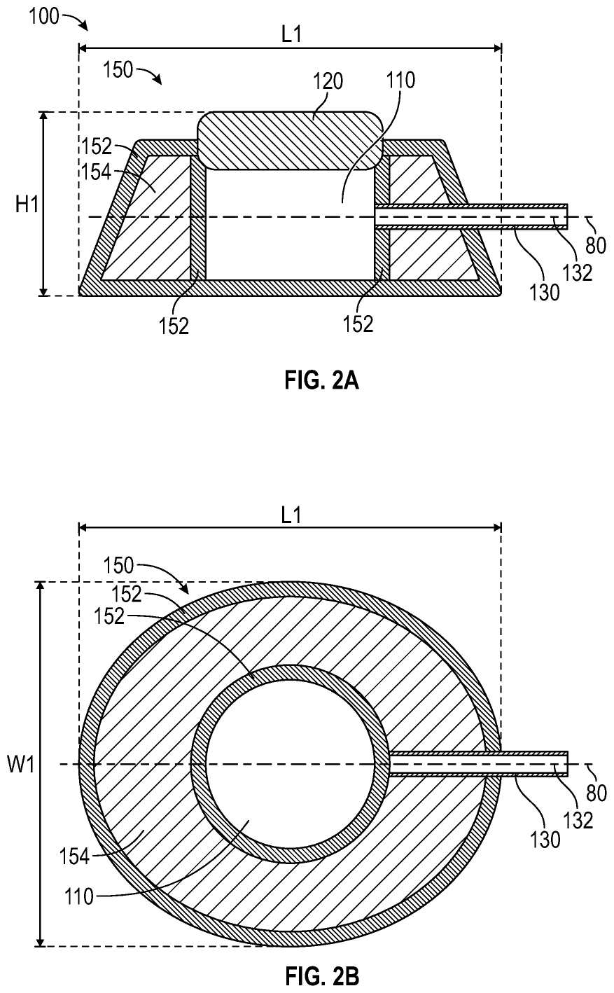

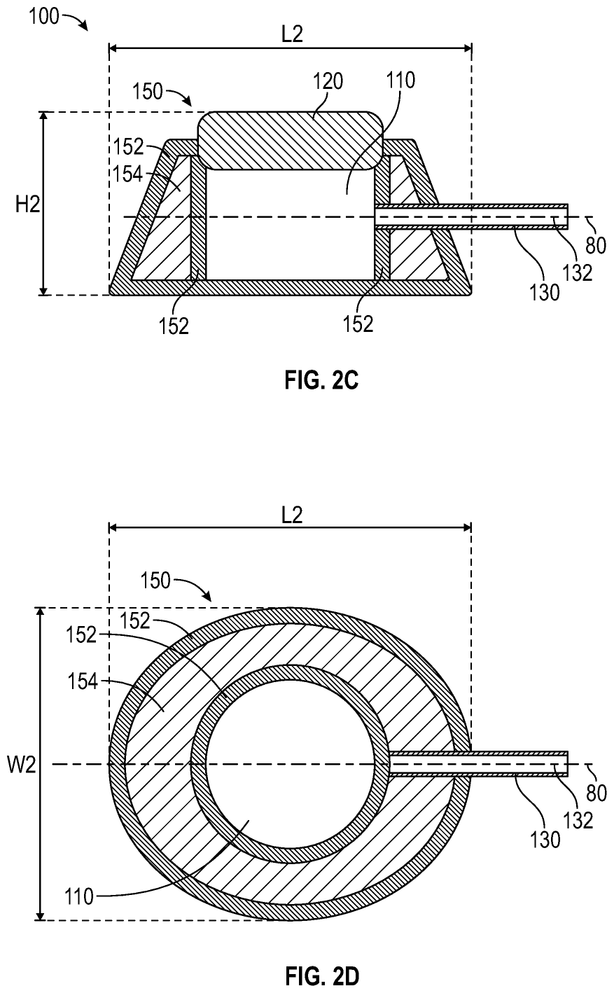

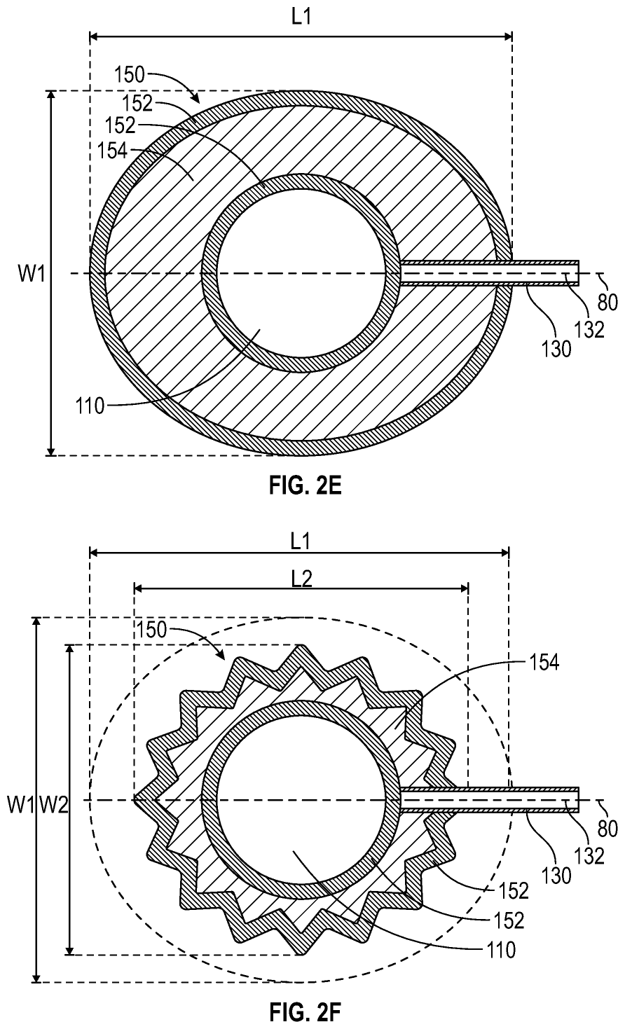

In an embodiment, as shown in FIGS. 2A-2D, the port body 150 can be configured to transition between an expanded configuration (FIGS. 2A-2B) and a collapsed configuration (FIGS. 2C-2D). In an embodiment, the material of the port body 150 in the expanded configuration can be in an austenite phase and the material of the port body 150 in the collapsed configuration can be in a martensite phase.

FIG. 2A shows a longitudinal, cross-sectional view of the port 100 in the expanded configuration. FIG. 2B shows a plan, cross-sectional view of the port 100 in the expanded configuration. FIG. 2C shows a longitudinal, cross-sectional view of the port 100 in the collapsed configuration. FIG. 2D shows a plan, cross-sectional view of the port 100 in the collapsed configuration.

In an embodiment, in the expanded configuration (FIGS. 2A-2B) the port body 150 can define a first port height (H1), a first port width (W1), or a first port length (L1), extending along the transverse, lateral, and longitudinal axes respectively. However, it will be appreciated that the height, width, or length can each extend along different axes. In an embodiment, the port body 150 in the expanded configuration can define a first port volume (V1) providing a first outer profile or size.

In an embodiment, in the collapsed configuration (FIGS. 2C-2D) the port body 150 can define a second port height (H2), a second port width (W2), a second port length (1.2), or a second port volume (V2) providing a second outer profile or size. In an embodiment, the second port height (H2) can be less than a first port height (H1). In an embodiment, the second port width (W2) can be less than a first port width (W1). In an embodiment, the second length port (1.2) can be less than a first port length (L1). In an embodiment, the second port volume (12) can be less than a first port volume (V1).

In an embodiment, as shown in FIGS. 2E-2F, the second, collapsed configuration of the port body 150 can include a folded configuration disposed on the outer profile. FIG. 2E shows a plan, cross-section view of the port 100 in the expanded configuration. FIG. 2F shows a plan, cross-section view of the port 100 in the collapsed configuration including a folded portion. In an embodiment, the folded portion of the port body can allow the outer surface of the port body 150 to be collapsed into a smaller profile, volume or size. In an embodiment, as shown in FIG. 2F the folded portion can include a plurality of pleats extending substantially vertically and allowing the outer surface of the port body 150 to be disposed within a small horizontal circumference. For reference, the horizontal circumference of the port body 150 in the expanded configuration is shown in dashed lines. In an embodiment, the folded portion can be formed out of the first material 152, one or more second materials 154, or combinations thereof.

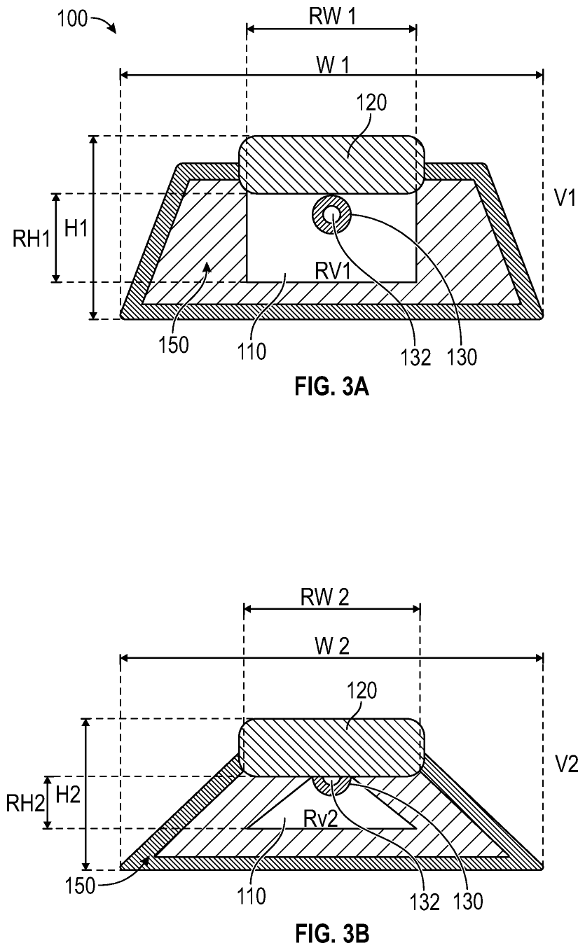

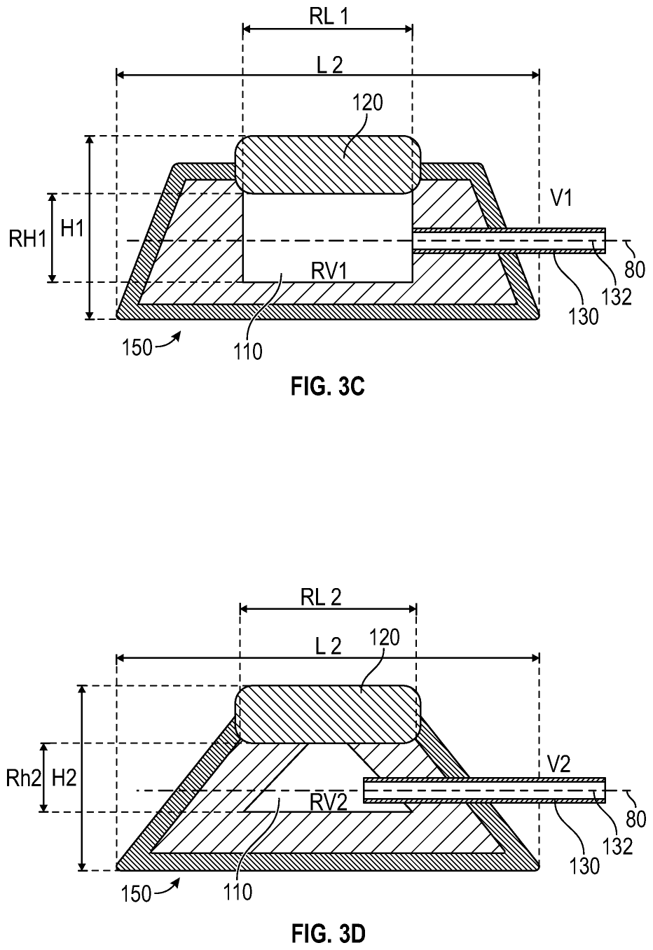

FIGS. 3A-3D shown an embodiment of the port 100 including a collapsible reservoir 110. FIG. 3A shows a lateral, cross-section view of the port in the expanded configuration. FIG. 3B shows a lateral, cross-section view of the port in the collapsed configuration. FIG. 3C shows a longitudinal, cross-section view of the port in the expanded configuration. FIG. 3D shows a longitudinal, cross-section view of the port in the collapsed configuration.

In an embodiment, in the expanded configuration the reservoir can define a first reservoir height (RH1), a first reservoir width (RW1), or a first reservoir length (RL1), extending along the transverse, lateral, and longitudinal axes respectively. However, it will be appreciated that the height, width, or length can each extend along different axes. In an embodiment, the reservoir 110 can define a first reservoir volume (RV1). In an embodiment, in the collapsed configuration the reservoir 110 can define a second reservoir height (RH2), a second reservoir width (RW2), a second reservoir length (RL2), or a second reservoir volume (RV2). In an embodiment, the second reservoir height (RH2) can be less than a first reservoir height (RH1). In an embodiment, the second reservoir width (RW2) can be less than a first reservoir width (RW1). In an embodiment, the second reservoir length (RL2) can be less than a first reservoir length (RL1). In an embodiment, the second reservoir volume (RV2) can be less than a first reservoir volume (RV1). In an embodiment, the second reservoir volume (RV2) of the reservoir 110 can define a zero volume, or a de minimis volume.

In an embodiment, the port body 150 can be exposed to a first temperature (e.g. body heat, or greater than or equal to 98.6° F. (37° C.)) and can provide the first configuration, e.g. the expanded configuration. The port body 150 can be exposed to a second temperature (e.g. less than body heat, or less than 98.6° F. (37° C.)), and can provide the second configuration, e.g. the collapsed configuration. In use when, the port 100 is disposed outside of the patient, the port 100 can define a collapsed configuration. Advantageously, the collapsed configuration can provide a smaller overall profile, size, or volume and can be disposed subcutaneously through a relatively smaller insertion site. Advantageously, the smaller insertion site can required fewer stitches, or no stitches, to close the insertion site leading to reduced scaring, improved recovery times, improved patient comfort and improved aesthetics. In an embodiment, when the port 100 is disposed subcutaneously, the port 100 is exposed to the first temperature, e.g. body heat, or greater than or equal to 98.6° F. (37° C.)) and can transition from the collapsed (second) configuration to the expanded (first) configuration. Advantageously, when the port 100 is disposed subcutaneously, the change in temperature can transition the port 100 to the expanded configuration ready for use.

In an embodiment, the port 100 in the expanded configuration can be plastically deformed, i.e. malleable, to a second, collapsed configuration while the port 100 is at the second temperature (e.g. less than 98.6° F. (37° C.)). The port 100 can remain in the collapsed configuration until the port 100 is placed subcutaneously. When placed subcutaneously, the change in temperature from the second temperature to the first temperature (e.g. greater than or equal to 98.6° F. (37° C.)) can cause the port 100 to transition from the second, collapsed configuration back to the first, expanded configuration. Advantageously, the port 100 can be plastically deformed to a smaller overall profile to allow for subcutaneous placement through a relatively smaller incision site. Once placed subcutaneously, the port 100 can be triggered by the change in temperature to transition from the collapsed configuration to the expanded configuration ready for use.

In an embodiment, the first temperature can be at a temperature that is greater than body heat, or greater than 98.6° F. (37° C.). As such, the port 100 can be transitioned from the expanded configuration to the collapsed configuration outside of the patient, as described herein. Once placed subcutaneously, the port 100 can remain in the collapsed configuration. In use, a clinician can apply a heat pad, or similar device, to a skin surface adjacent to the location of the port 100 which is placed subcutaneously. The heat pad can warm the port to a temperature greater than body heat or greater than 98.6° F. (37° C.) and can transition the port 100 from the collapsed configuration to the expanded configuration ready for use. Once the treatment has been completed, the port 100 can be allowed to cool back down to body heat and the port 100 can transition to the collapsed configuration. In an embodiment, the port 100 can be allowed to cool back down to body heat and the port 100 can be plastically deformed by palpation through the skin to a collapsed configuration providing a lower profile. Advantageously, the port 100 can maintain the collapsed configuration after subcutaneous placement between uses providing a lower profile, reducing stretching of the skin, reducing scaring, and improving patient comfort and aesthetics. When the port 100 is required to be accessed the port 100 can be selectively transitioned to the expanded configuration by the application of heat.

In an embodiment, the port 100 can be plastically deformed to reduce in size along a first axis and expand in size along a second axis, extending at an angle to the first axis. As such, the port body 150 can plastically deform to reduce a cross-sectional area in a first plane and fit through a relatively smaller incision site than would otherwise be required in the expanded configuration. For example, the port body 150 can be configured to plastically deform and reduce in size along one of the transverse or lateral axes, and can expand along the longitudinal axis, to allow a cross-sectional area of the port body 150 to reduce along the laterally vertical plane. As such, the port 100 can fit through a relatively smaller insertion site than would otherwise be required in the expanded configuration.

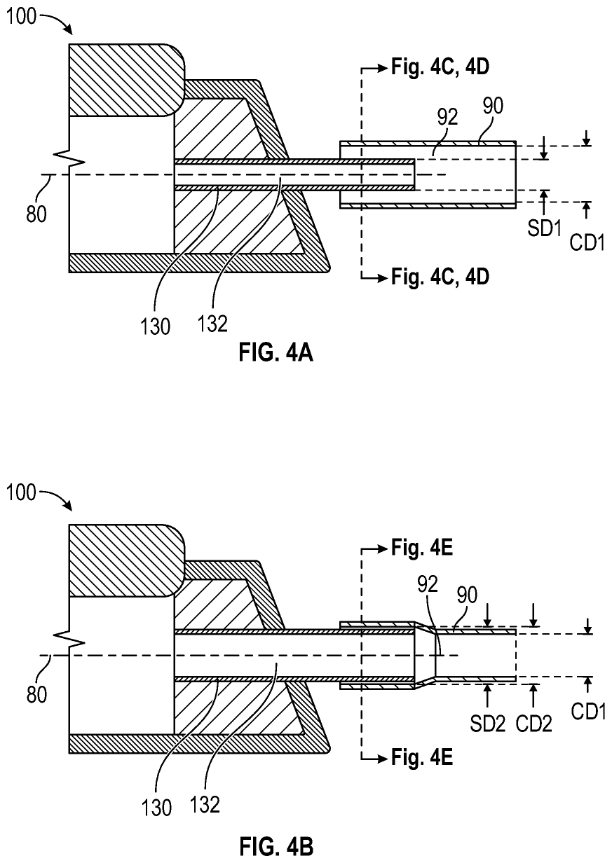

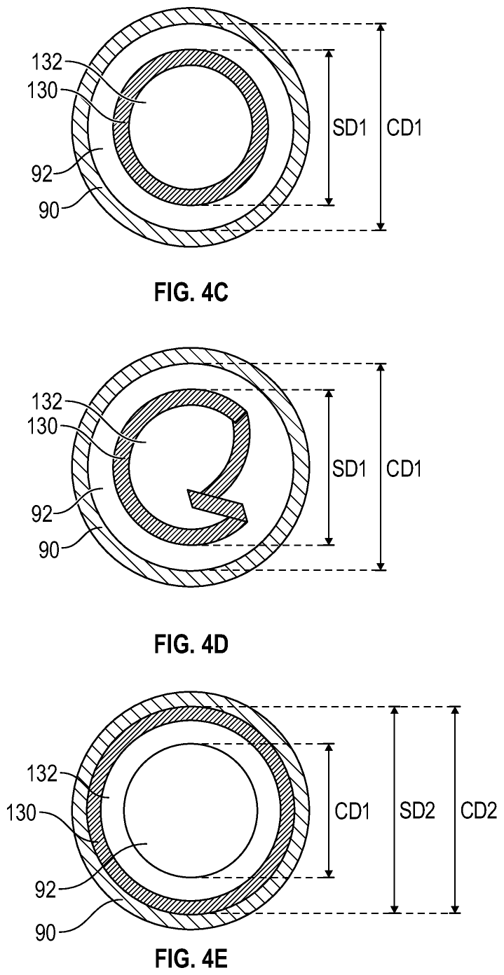

In an embodiment, as shown in FIGS. 4A-4E, a port stem 130 can include a first material, e.g. a shape memory material, and can be configured to transition between a collapsed configuration (FIGS. 4A, 4C, 4D) and an expanded configuration (FIGS. 4B, 4E). FIGS. 4A-4B show a longitudinal cross-section view. FIGS. 4C-4E show a lateral cross-section view through the port stem 130 in a distal direction, i.e. towards the catheter 90. In an embodiment, the material of the port stem 130 in the expanded configuration can be in an austenite phase and the material of the port stem 130 in the collapsed configuration can be in a martensite phase.

As shown in FIGS. 4A, 4C-4D, in the collapsed configuration, the port stem 130 can define a first outer stem diameter (SD 1). The first stem diameter (SD 1) can be less than a first inner lumen diameter (CD 1) of a catheter 90 in a relaxed state. As shown in FIGS. 4B and 4E, in the expanded configuration, the port stem 130 can define a second outer stem diameter (SD 2). The second stem diameter (SD 2) can be the same as or greater than the first inner lumen diameter (CD 1) of a catheter 90 in a relaxed state. In an embodiment, the port stem 130 in the collapsed configuration can include a folded portion (FIG. 4D) configured to allow the port stem 130 to transition between the first outer stem diameter (SD 1) of the collapsed configuration, and the second outer stem diameter (SD 2) of the expanded configuration.

In use, the catheter 90 can be coupled to the port stem 130 by slidably engaging the port stem 130 with the catheter lumen 92, while the port stem 130 is in the collapsed configuration. The port stem 130 can then be transitioned from the collapsed configuration (FIGS. 4A, 4C-4D) to the expanded configuration (FIGS. 4B, 4E). In an embodiment, the port stem 130 can be transitioned from the expanded configuration to the collapsed configuration by a change in temperature, and/or by plastic deformation, as described herein. In an embodiment, the port stem 130 can be transitioned from the collapsed configuration to the expanded configuration by a change in temperature, from being placed subcutaneously, as described herein. In the expanded configuration, the port stem 130 can define a second outer diameter (SD 2) greater than then inner lumen diameter of the catheter in the relaxed state (CD 1). As such, the port stem 130 can stretch a portion of the catheter to a second inner lumen diameter (CD 2) and provide a fluid tight seal therebetween.

Advantageously, the port stem 130 can provide a secure interference fit between the port stem and the catheter lumen. Further the catheter can be slidably engaged with the port stem 130 before transitioning to the expanded configuration, and requires relatively little columnar force, mitigating slippage within the confined wetted environment of the tissue pocket. Once engaged the port stem can be transitioned to the expanded configuration to provide a tight, interference fit therebetween.

In an embodiment, a first temperature can transition the port 130 from the first, expanded configuration to the second, collapsed configuration, and a second temperature can transition the port 130 from the second collapsed configuration to the first expanded configuration. In an embodiment, the port 130 can be plastically deformed from the first, expanded configuration to the second, collapsed configuration while at the first temperature. The second temperature can then transition the port stem 130 from the second, collapsed configuration, to the first, expanded configuration.

In an embodiment, the first temperature can be less than body heat, or less than 98.6° F. (37° C.)). The second temperature can be greater than or equal to body heat, or 98.6° F. (37° C.)). As such, the port stem 130 disposed externally to the patient can be exposed to the first temperature and can be transitioned to, or plastically deformed to, the second collapsed configuration. The port 100 can then be disposed subcutaneously and the port stem 130 can be slidably engaged with the catheter lumen 92. The port stem 130 can then be exposed to the second temperature, i.e. greater than or equal to body heat, or 98.6° F. (37° C.)), and can transition the port stem 130 from the collapsed configuration to the expanded configuration, providing a fluid tight seal therebetween.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A subcutaneous access system, comprising:
a catheter defining a lumen; and
a port, comprising:
a port stem configured to engage a catheter and provide fluid communication therewith;
a body including a shape memory material and defining a reservoir in fluid communication with the port stem, the body transitionable between an expanded configuration and a collapsed configuration, the collapsed configuration defining a smaller overall profile; and
a needle penetrable septum disposed over the reservoir and configured to provide percutaneous access thereto.

2. The subcutaneous access system according to claim 1, wherein the port stem includes the shape memory material and is configured to transition between an expanded configuration and a collapsed configuration.

3. The subcutaneous access system according to claim 2, wherein the port stem in the collapsed configuration defines a first outer stem diameter, and the port stem in the expanded configuration defines a second outer stem diameter, the second outer stem diameter being larger than the first outer stem diameter.

4. The subcutaneous access system according to claim 3, wherein the first outer stem diameter is less than an inner lumen diameter of the catheter in a relaxed state and the second outer stem diameter is greater than the inner lumen diameter of the catheter in the relaxed state.

5. The subcutaneous access system according to claim 1, wherein the shape memory material includes a metal, composite, or an alloy and includes one of nickel, titanium, zinc, copper, gold, iron, aluminum, a copper-aluminum-nickel alloy, a nickel-titanium alloy, or Nitinol.

6. The subcutaneous access system according to claim 1, wherein the body in the expanded configuration defines one of a first port height, a first port width, or a first port length, and wherein the body in the collapsed configuration defines one of a second port height, a second port width, or a second port length.

7. The subcutaneous access system according to claim 6, wherein one of the second port height is less than the first port height, the second port width is less than the first port width, or the second port length is less than the first port length.

8. The subcutaneous access system according to claim 1, wherein the body in the expanded configuration defines a first port volume, and the body in the collapsed configuration defines a second port volume, the second port volume being less than the first port volume.

9. The subcutaneous access system according to claim 1, wherein the reservoir in the expanded configuration defines one of a first reservoir height, a first reservoir width, or a first reservoir length, and wherein the reservoir in the collapsed configuration defines one of a second reservoir height, a second reservoir width, or a second reservoir length.

10. The subcutaneous access system according to claim 9, wherein one of the second reservoir height is less than the first reservoir height, the second reservoir width is less than the first reservoir width, or the second reservoir length is less than the first reservoir length.

11. The subcutaneous access system according to claim 1, wherein the reservoir in the expanded configuration defines a first reservoir volume, and the reservoir in the collapsed configuration defines a second reservoir volume, the second reservoir volume being less than the first reservoir volume.

12. The subcutaneous access system according to claim 1, wherein the body at a first temperature can transition from the collapsed configuration to the expanded configuration, and the body at a second temperature can transition from the expanded configuration to the collapsed configuration.

13. The subcutaneous access system according to claim 12, wherein the first temperature is equal to or greater than 98.6° F. (37° C.) and the second temperature is less than 98.6° F. (37° C.).

14. The subcutaneous access system according to claim 12, wherein the first temperature is greater than 98.6° F. (37° C.) and the second temperature is equal to or less than 98.6° F. (37° C.).

15. The subcutaneous access system according to claim 12, wherein the body at the second temperature is configured to be plastically deformable.

16. The subcutaneous access system according to claim 12, wherein the body at the first temperature is in an austenite phase, and the body at the second temperature is in a martensite phase.

17. The subcutaneous access system according to claim 1, wherein the body in the collapsed configuration includes a folded portion disposed on an outer surface of the body.

18. The subcutaneous access system according to claim 17, wherein the folded portion includes a plurality of pleats disposed on an outer surface of the body and configured to allow the body to transition between the expanded configuration and the collapsed configuration.

19. The subcutaneous access system according to claim 1, wherein the body includes one of a frame, a shell, a honeycomb or an exoskeleton morphology formed of the shape memory material.

20. The subcutaneous access system according to claim 19, further including a second material different from the shape memory material and disposed thereon to form a continuous outer profile.

21-40. (canceled)
